(19) United States
(12) Patent Application Publication
(10) Pub. No.: US 2014/0235508 A1
(43) Pub. Date: Aug. 21, 2014

(54) NUCLEIC ACID LINKER

(71) Applicants: National University Corporation Saitama University, Saitama-shi (JP); Nikon Corporation, Tokyo (JP)

(72) Inventors: Naoto Nemoto, Saitama-shi (JP); Shingo Ueno, Kawaguchi-shi (JP); Hirofumi Shiono, Fujisawa-shi (JP)

(73) Assignees: National University Corporation Saitama University, Saitama-shi (JP); Nikon Corporation, Tokyo (JP)

(21) Appl. No.: 14/265,938

(22) Filed: Apr. 30, 2014

Related U.S. Application Data
(63) Continuation of application No. PCT/JP2012/078488, filed on Nov. 2, 2012.

(30) Foreign Application Priority Data
Nov. 4, 2011 (JP) 2011-242790

(51) Int. Cl. C07K 17/10

(52) U.S. Cl. CPC C07K 17/10
USPC 506/18; 536/23.1; 530/358

(57) ABSTRACT

A nucleic acid linker is for producing a complex of mRNA and a protein encoded by that mRNA, comprising one 3′-terminal region and two branched 5′-terminal regions, wherein the 3′-terminal region comprises a single-stranded polynucleotide segment able to hybridize with the sequence on the 3′-terminal side of the mRNA, and an arm segment that branches off from the single-stranded polynucleotide segment and has a linking segment with the protein on the terminal thereof, and one of the two 5′-terminal regions has a binding site with the 3′-terminal of the mRNA.

### CROSS-REFERENCE TO RELATED APPLICATION

This is a Continuation Application of International Application No. PCT/JP2012/078488, filed Nov. 2, 2012, which claims priority to Japanese Patent Application No. 2011-242790 filed in Japan on Nov. 4, 2011. The contents of the aforementioned applications are incorporated herein by reference.

### BACKGROUND

The present invention relates to a nucleic acid linker.

New functional proteins are expected to contribute to various applications in the field of biotechnology, such as in pharmaceuticals, detergents, food processing, reagents for research and development, clinical analyses as well as bioenergy and biosensors.

Although protein engineering techniques, consisting of using human intellect to design proteins based on protein structural information, have been primarily used when acquiring new functional proteins, since screening methods more efficient than those used in the past are required to acquire more useful proteins, expectations are being placed on molecular evolutionary engineering techniques that consist of randomly repeating modification and screening of protein molecular structure.

The cDNA display method, which is a type of molecular evolutionary engineering technique, is a method for associating genotype and phenotype, and consists of the use of a nucleic acid linker to link a protein (phenotype) with mRNA encoding the protein and reverse-transcribed cDNA (genotype). Since the mRNA/cDNA-protein linkage structure is extremely stable, screening can be carried out in various environments by using this nucleic acid linker.

The cDNA display method is characterized by the presence of puromycin in a nucleic acid linker that links a protein with a polynucleotide that encodes that protein (see Japanese Patent No. 4318721).

Puromycin is a protein synthesis inhibitor having a structure that resembles the 3′-terminal of aminoacyl-tRNA, and under prescribed conditions, specifically covalently bonds to the C-terminal of protein during elongation on a ribosome.

Methods for screening useful proteins using the cDNA display method consist of the series of steps described below.

First, a nucleic acid linker containing puromycin is coupled to mRNA, protein is synthesized from the mRNA using a cell-free translation system, and the synthesized protein and mRNA encoding that protein are linked through puromycin to form a complex (mRNA-nucleic acid linker-protein complex) (see Nemoto, et al., FEBS Lett., Vol. 414, pp. 405-408, 1997).

Next, a library of this mRNA-nucleic acid linker-protein complex is prepared, the prepared mRNA-nucleic acid linker-protein complex is reverse-transcribed with reverse transcriptase to synthesize cDNA, and this synthesized cDNA is used to prepare an mRNA/cDNA-nucleic acid linker-protein complex library, followed by selecting a protein having a desired function. The protein can be identified by analyzing the base sequence of the cDNA in the selected mRNA/cDNA-nucleic acid linker-protein complex. Reverse transcription may also be carried out prior to protein selection (see Yamaguchi, et al., Nucleic Acids Res., Vol. 37, p. e108, 2009).

A protein array, in which a library of the aforementioned mRNA (or mRNA/cDNA)-nucleic acid linker-protein complex is immobilized on a substrate, is important as a tool for acquiring functional protein in a short period of time by comprehensive analysis. Known examples of nucleic acid linkers used for such comprehensive analysis are shown in FIG. 13 (see Japanese Unexamined Patent Application, First Publication No. 2004-97213). In a nucleic acid linker 100 shown in FIG. 13 (A), the 5′-terminal side of a single-stranded DNA sequence forms a complementary double-stranded sequence through a loop region, and has a solid phase binding site in the loop region for binding to a substrate. In addition, in a nucleic acid linker 101 shown in FIG. 13(B), two single-stranded DNA sequences having mutually complementary sequences on the 5′-side thereof form a double-stranded sequence through those complementary sequences, and there is a solid phase binding site on the 3′-terminal of one of the two single-stranded DNA sequences.

### SUMMARY

A nucleic acid linker is equivalent to a “linking segment” for linking mRNA and protein using a cell-free translation system. Since conventional nucleic acid linkers like those described above are designed for in vitro selection, although they have a solid phase binding site, they merely serve as one of the steps for improving the efficiency of synthesizing mRNA/cDNA-protein complexes. Consequently, they have multiple problems from the viewpoint of molecular manipulation techniques in the case of using a screening system such as a protein array in which an mRNA-protein complex is immobilized on a solid phase.

More specifically, these problems include 1) the lack of an efficient desorption mechanism enabling release of the mRNA/cDNA-protein complex from the solid phase, and 2) the lack of a spacer for maintaining an interval with the solid phase in order to avoid the effects of non-specific adsorption and optical characteristics of the solid phase substrate.

As a result of conducting extensive studies, the inventors of the present invention found that problems can be solved by introducing a branched chain into the 5′-side of a nucleic acid linker. Embodiments of the present invention provide that described in the following (1) to (15).

(1) The nucleic acid linker in one embodiment of the present invention is a nucleic acid linker for producing a complex of mRNA and a protein encoded by that mRNA, comprising:

one 3′-terminal region, and

two branched 5′-terminal regions; wherein,

the 3′-terminal region comprises a single-stranded polynucleotide segment able to hybridize with the sequence on the 3′-terminal side of the mRNA, and

an arm segment that branches off from the single-stranded polynucleotide segment and has a linking segment with the protein on the terminal thereof, and

one of the two 5′-terminal regions has a binding site with the 3′-terminal of the mRNA.

(2) In the nucleic acid linker in one embodiment of the present invention, the other of the two 5′-terminal regions can have a solid phase binding site on the 5′-terminal thereof.

(3) In the nucleic acid linker of one embodiment of the present invention, the other of the two 5′-terminal regions can contain a cleavage site.

(4) In the nucleic acid linker of one embodiment of the present invention, one of the 3′-terminal region and the 5′-terminal region can contain a cleavage site.

(5) In the nucleic acid linker of one embodiment of the present invention, the linking segment with the protein can have puromycin, a 3′-N-aminoacyl puromycin aminonucleoside or 3′-N-aminoacyl adenosine aminonucleoside bound to the end of the arm segment.

(6) In the nucleic acid linker of one embodiment of the present invention, one of the two 5′-terminal regions and the 3′-terminal region can form a loop region.

(7) In the nucleic acid linker of one embodiment of the present invention, one of the two 5′-terminal regions and the 3′-terminal region respectively can contain a cleavage site.

(8) The nucleic acid linker in one embodiment of the present invention is a nucleic acid linker for producing a complex of mRNA and a protein encoded by that mRNA, provided with:

a 3′-terminal region containing a single-stranded polynucleotide segment able to hybridize with the sequence on the 3′-terminal side of the mRNA,

two branched 5′-terminal regions, and

an arm segment having a linking segment with the protein on the terminal thereof; wherein,

at least one of the two 5′-terminal regions has a spacer region containing a solid phase binding site on the 5′-terminal thereof.

(9) In the nucleic acid linker in one embodiment of the present invention, the protein can compose anyone of an enzyme, antibody, antigen, aptamer and peptide.

(10) In the nucleic acid linker in one embodiment of the present invention, the arm segment can have a labeling substance.

(11) The mRNA-nucleic acid linker-protein complex in one embodiment of the present invention is obtained by linking the mRNA and a protein encoded by that mRNA through the previously described nucleic acid linker.

(12) The mRNA/cDNA-nucleic acid linker-protein complex in one embodiment of the present invention is obtained by linking an mRNA/cDNA complex, composed of the mRNA and cDNA complementary to the mRNA, and a protein encoded by that mRNA through the previously described nucleic acid linker.

(13) The method for producing the mRNA-nucleic acid linker-protein complex in one embodiment of the present invention has:

(a) a step for annealing the mRNA and the nucleic acid linker,

(b) a step for ligating the 3′-terminal of the mRNA and the 5′-terminal of the nucleic acid linker, and

(c) a step for preparing the mRNA-nucleic acid linker-protein complex, in which the C-terminal of the protein is bound to a linking segment with the protein of the nucleic acid linker, by synthesizing the protein from the mRNA using a cell-free protein translation system.

(14) The method for producing the mRNA/cDNA-nucleic acid linker-protein complex in one embodiment of the present invention has:

(a) a step for annealing the mRNA and the nucleic acid linker,

(b) a step for ligating the 3′-terminal of the mRNA and the 5′-terminal of the nucleic acid linker,

(c) a step for preparing an mRNA-nucleic acid linker-protein complex, in which the C-terminal of the protein is bound to a linking segment with the protein of the nucleic acid linker, by synthesizing the protein from the mRNA using a cell-free protein translation system, and

(d) a step for synthesizing cDNA from the mRNA-nucleic acid linker-protein complex by reverse transcription.

(15) The protein array in one embodiment of the present invention is obtained by immobilizing the previously described protein complex on a substrate.

### BRIEF DESCRIPTION OF THE DRAWINGS

FIG. 1 is a drawing showing one aspect of a nucleic acid linker used in one embodiment.

FIG. 2 is a drawing showing one aspect of a nucleic acid linker used in one embodiment.

FIG. 3 is a drawing showing one aspect of a nucleic acid linker used in one embodiment.

FIG. 4 indicates the results of electrophoresis in an example.

FIG. 5 indicates the results of electrophoresis in an example.

FIG. 6 indicates the results of electrophoresis in an example.

FIG. 7 indicates the results of electrophoresis in an example.

FIG. 8 indicates the results of electrophoresis in an example.

FIG. 9 indicates the results of electrophoresis in an example.

FIG. 10 indicates the results of electrophoresis in an example.

FIG. 11 is a schematic drawing showing the product of covalently bonding BDA (B-domain of Protein A) mRNA and a nucleic acid linker in an example.

FIG. 12 indicates the results of electrophoresis in an example.

FIG. 13 is a drawing showing one aspect of a nucleic acid linker of the prior art.

### DETAILED DESCRIPTION

### <<Nucleic Acid Linker>>

### First Embodiment

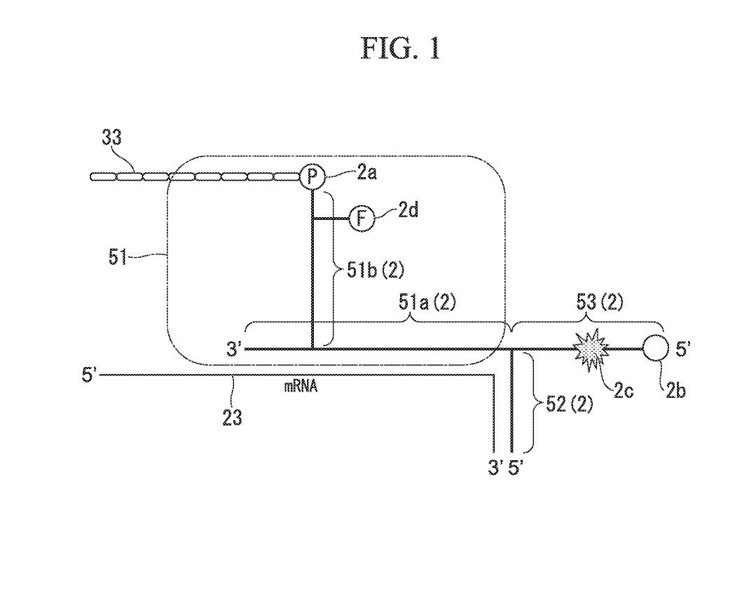

The nucleic acid linker 2 of the present embodiment is a linker for linking an mRNA 23 and a protein 33 encoded thereby. The following provides an explanation of the structure of the nucleic acid linker of the present embodiment with reference to FIG. 1.

In FIG. 1, P indicates puromycin, and F indicates fluorescein.

The nucleic acid linker 2 is composed of one 3′-terminal region 51 and two branched 5′-terminal regions (one region 52 and other region 53).

The 3′-terminal region 51 comprises a single-stranded polynucleotide segment 51a, which is able to hybridize with the sequence on the 3′-terminal side of the mRNA 23 to be screened, and an arm segment 51b, which branches from the single-stranded polynucleotide segment 51a and has a linking segment 2a with the protein 33 on the terminal thereof.

The single-stranded polynucleotide segment 51a may be DNA or a nucleic acid derivative such as a polynucleopeptide (PNA), and is preferably modified DNA imparted with nuclease resistance. Any modified DNA known in the art may be used as modified DNA, examples of which include DNA having an internucleoside bond such as a phosphorothioate and DNA having a sugar modification such as 2′-fluoro, 2′-O-alkyl.

The arm segment 51b functions as a spacer that maintains a desired distance between the mRNA 23 and the protein linking segment 2a. The 5′-terminal of the arm segment 51b bonds to the single-stranded polynucleotide segment 51a at a location on the 3′-terminal side of the single-stranded polynucleotide segment 51a, while the 3′-terminal of the arm segment 51b has the protein linking segment 2a.

Linking between the single-stranded polynucleotide segment 51a and the arm segment 51b can be carried out by crosslinking between a modified nucleotide present at a linking location on the single-stranded polynucleotide segment 51a (such as a nucleotide in which an amino group is introduced into a base moiety through a spacer) and a modified nucleotide present on the end of the arm segment 51b (such as a nucleotide having a thiol on the 5′-terminal thereof) using a bifunctional reagent.

As will be subsequently described, in the case mRNA encoding a protein to be screened is required to be reverse-transcribed, the 5′-terminal of the arm segment 51b preferably forms a T-shaped structure by bonding with the single-stranded polynucleotide segment 51a at a location several bases towards the 5′-side from the 3′-terminal of the single-stranded polynucleotide segment 51a. This is because the 3′-terminal of the single-stranded polynucleotide segment 51a functions as a primer during reverse transcription.

The single-stranded polynucleotide segment 51a or the arm segment 51b, excluding the 3′-terminal thereof, may be labeled using a labeling substance. The labeling substance is suitably selected from a fluorescent dye or radioactive substance and the like.

In the present embodiment, as shown in FIG. 1, the arm segment 51a is modified with fluorescein 2d, excluding the 3′-terminal thereof. The nucleic acid linker 2 is fluorescent-labeled as a result of this modification, thereby enabling an mRNA 23-nucleic acid linker 2 complex or mRNA 23-nucleic acid linker 2-protein 33 complex to be easily detected.

The linking segment 2a with the protein 33 is present on the 3′-terminal of the arm segment 51b. The protein linking segment 2a refers to a structure having the property of specifically bonding to the C-terminal of the protein 33 during elongation on a ribosome under prescribed conditions, and a typical example thereof is puromycin.

Puromycin is a protein synthesis inhibitor having a structure that resembles the 3′-terminal of aminoacyl-tRNA. Any arbitrary substance can be used for the linking segment 2a with the protein 33 provided it has a function that allows it to specifically bond to the C-terminal of the protein 33 during elongation, and puromycin derivatives such as 3′-N-aminoacyl puromycin aminonucleoside (PANS-amino acid) or 3′-N-aminoacyl adenosine aminonucleoside (AANS-amino acid) can be used.

Examples of PANS-amino acids include PANS-Gly in which the amino acid moiety is glycine, PANS-Val in which it is valine, PANS-Ala in which it is alanine, and PANS-amino acid mixtures in which the amino acid moieties correspond to each amino acid in all amino acids.

Examples of AANS-amino acids include AANS-Gly, in which the amino acid moiety is glycine, AANS-Val in which it is valine, AANS-Ala in which it is alanine, and AANS-amino acid mixtures in which the amino acid moieties correspond to each amino acid in all amino acids.

Examples of amino acyl-tRNA 3′-terminal analogues able to be used preferably other than puromycin include ribocytidyl puromycin (rCpPur), deoxycytidyl puromycin (dCpPur) and deoxyuridyl puromycin (dUpPur).

The arm segment 51b may be composed of nucleic acids or nucleic acid derivatives provided it functions as a spacer, and may be composed of a polymer such as polyethylene glycol.

Modifications for enhancing the stability of puromycin or a label for detecting a complex may be further added to the arm segment 51b.

The 5′-terminal region is branched into two regions consisting of one region 52 and other region 53. The one region 52 preferably forms a T-shaped structure by branching from the boundary between the single-stranded polynucleotide segment 51a of the 3′-terminal region 51 and the other region 53. A modified nucleotide amidite or branching phosphate group amidite capable of synthesizing branched chains from a base moiety through a spacer is used to synthesize this branched segment in the form of the one region 52.

The 5′-terminal of the one region 52 is preferably ligated with the 3′-terminal of the mRNA 23 in order to strengthen bonding with the single-stranded polynucleotide segment 51a able to hybridize with the mRNA 23.

The other region 53 of the nucleic acid linker 2 of the present embodiment preferably contains a cleavage site 2c. Examples of the cleavage site 2c include a photocleavage site and a single-stranded nucleic acid cleaving enzyme cleavage site.

The mRNA 23 associated with the protein 33 (or cDNA obtained by reverse transcription of the mRNA 23) can be recovered due to the presence of the cleavage site 2c.

A photocleavage site refers to a group having the property of being cleaved when irradiated with light such as ultraviolet light, and examples of products using this group include PC Linker Phosphoramidite (Glen Research), a composition for nucleic acid photocleavage containing fullerene (Composition for Nucleic Acid Photocleavage: Japanese Unexamined Patent Application, First Publication No. 2005-245223), and strand breakage by photolysis (SBIP method).

A commercially available product in the art or any known group, such as a nitrobenzyl group, may be used as a photocleavage site.

In addition, a single-stranded nucleic acid cleaving enzyme cleavage site refers to a nucleic acid group able to be cleaved by a single-stranded nucleic acid cleaving enzyme such as deoxyribonuclease or ribonuclease, and includes nucleotides and derivatives thereof, such as deoxyinosine recognized by endonuclease V.

The other region 53 of the nucleic acid linker 2 of the present embodiment preferably has a solid phase binding site 2b on the 5′-terminal thereof.

In addition to methods utilizing avidin-biotin bonding, a method consisting of modifying the nucleic acid linker 2 with a functional group such as an amino group, formyl group or SH group and treating the surface of the solid phase with a silane coupling agent having an amino group, formyl group or epoxy group and the like, or a method that utilizes gold-thiol bonding, can be preferably used for immobilization of the nucleic acid linker 2, while a method that utilizes avidin-biotin bonding is particularly preferable.

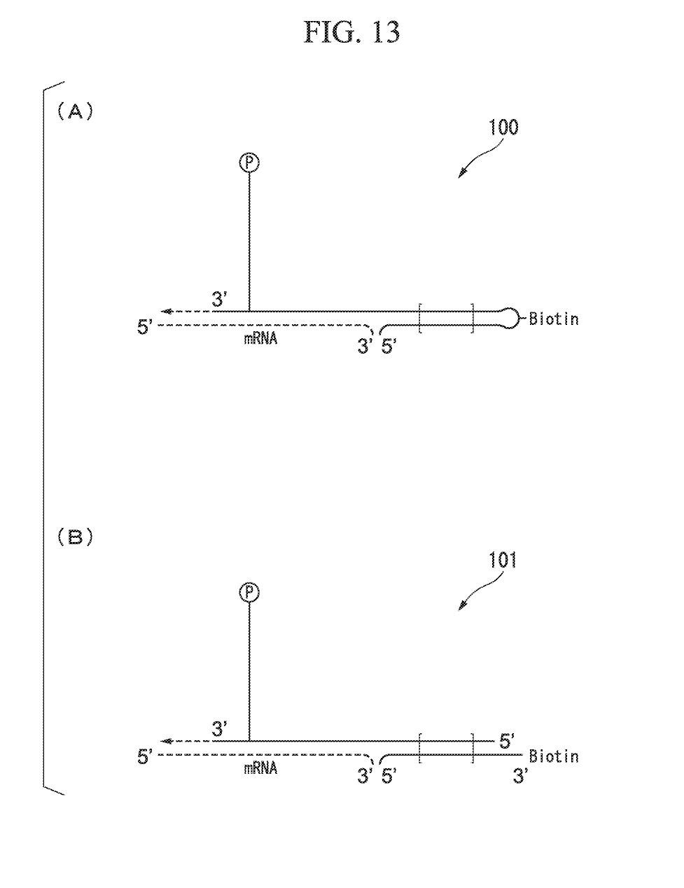

The nucleic acid linker 2 of the present embodiment eliminates the need to prepare two single-stranded DNA sequences in the manner of the nucleic acid linker 101 shown in FIG. 13(B) as a result of having the one region 52 in the form of a branched chain.

Since the nucleic acid linker 2 of the present embodiment has the other region 53, distance can be created between the solid phase and the cleavage site 2c by extending the base sequence of the 5′-terminal that composes the other region 53.

As a result, in the case of ligating the nucleic acid linker 2 immobilized on a substrate with the mRNA 23 on the substrate, there is no risk of having an effect on ligation efficiency attributable to the distance between the substrate and the nucleic acid linker 2 being short.

In addition, in the case of using the nucleic acid linker 2 having a nitrobenzyl group for the cleavage site 2c and using a gold substrate for the solid phase, for example, there is the risk of the gold substrate absorbing light energy required to cleave the nitrobenzyl group if the distance between the gold substrate and the nitrobenzyl group is short. In the present embodiment, this risk is eliminated, thereby making it possible to recover the mRNA 23 associated with the protein 33 (or cDNA obtained by reverse transcription of the mRNA 23) by efficiently cleaving the nucleic acid linker 2 by photoirradiation.

In addition, the other region 53 can be modified as desired in the nucleic acid linker 2 of the present embodiment. Namely, the region between the nucleic acid linker and the solid phase can be modified as desired.

In this manner, the nucleic acid linker 2 of the present embodiment enables highly functional molecular manipulation.

In addition, at least one of the aforementioned two 5′-terminal regions preferably has a spacer region containing a solid phase binding site on the 5′-terminal thereof. The nucleic acid linker of the present embodiment is used to produce a complex of mRNA and a protein encoded by that mRNA. As a result of having the aforementioned spacer region, the degree of freedom of the steric structure of the protein linked to the nucleic acid linker is thought to be ensured, and the efficiency of translation from mRNA bound to the nucleic acid linker to a protein is thought to increase.

In the case of considering translation efficiency in particular, the length of the aforementioned spacer region is preferably 10 nm or more, more preferably 15 nm or more and even more preferably 20 nm or more in consideration of the size of ribosomes used in translation.

In addition, the aforementioned protein preferably composes any one of an enzyme, antibody, antigen, aptamer and peptide.

### Second Embodiment

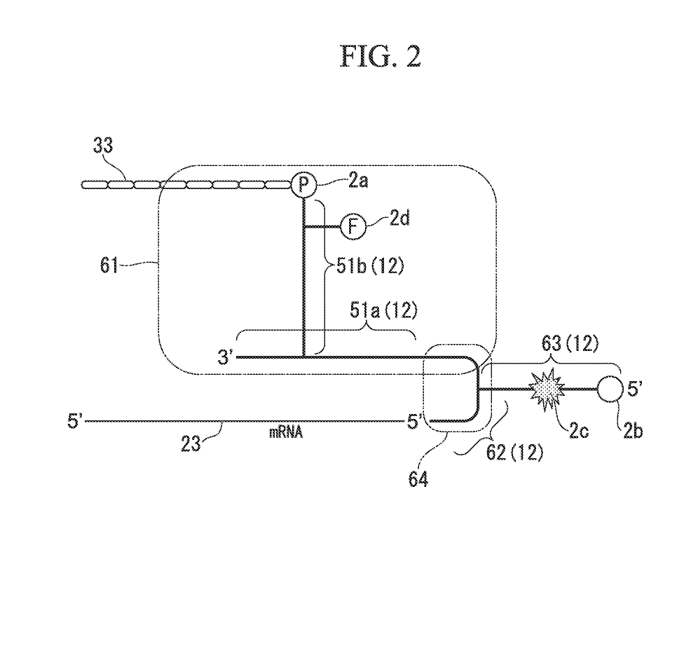

The following provides an explanation of the structure of a nucleic acid linker 12 of the present embodiment with reference to FIG. 2.

In FIG. 2, the same reference symbols are used to indicate those constituent elements that are the same as those shown in the schematic drawing of the nucleic acid linker 2 of FIG. 1, and an explanation thereof is omitted.

The nucleic acid linker 12 is composed of one 3′-terminal region 61 and two branched 5′-terminal regions (consisting of one region 62 and other region 63).

The one region 62 and the 3′-terminal region 61 form a loop region 64.

The 5′-terminal region is divided into two branched regions, consisting of the one region 62 and the other region 63. The other region 63 preferably forms a T-shaped structure by branching from the loop region 64. A modified nucleotide amidite or branching phosphate group amidite, capable of synthesizing branched chains from a base moiety through a spacer, is used to synthesize this branched segment in the form of the other region 63.

The 5′-terminal of the one region 62 is preferably ligated with the 3′-terminal of the mRNA 23 to strengthen the bond with the single-stranded polynucleotide segment 51a able to hybridize with the mRNA 23.

### Third Embodiment

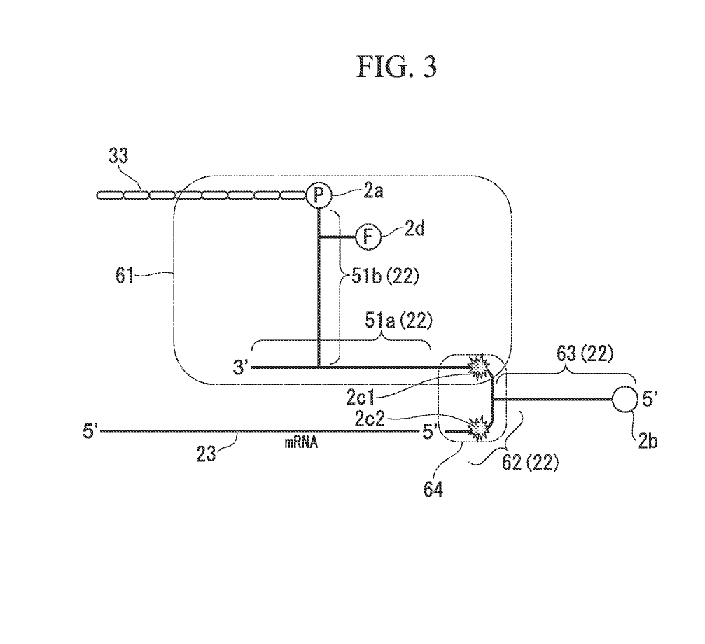

The following provides an explanation of the structure of a nucleic acid linker 22 of the present embodiment with reference to FIG. 3.

In FIG. 3, the same reference symbols are used to indicate those constituent elements that are the same as those shown in the schematic drawings of the nucleic acid linker 2 of FIG. 1 and the nucleic acid linker 12 of FIG. 2, and an explanation thereof is omitted.

In the nucleic acid linker 22 of the present embodiment, the 3-terminal region 61 and the one region 62 of the 5′-terminal region respectively contain a cleavage site 2c1 and a cleavage site 2c2. Examples of the cleavage sites 2c1 and 2c2 include photocleavage sites and single-stranded nucleic acid cleaving enzyme cleavage sites in the same manner as in the first embodiment.

<<mRNA-Nucleic Acid Linker-Protein Complex>>

An mRNA-nucleic acid linker-protein complex is produced using the nucleic acid linker of the present embodiment.

A method for producing the mRNA-nucleic acid linker-protein complex comprises:

(a) a step for annealing the mRNA and the nucleic acid linker,

(b) a step for ligating the 3′-terminal of the mRNA and the 5′-terminal of the nucleic acid linker, and

(c) a step for preparing the mRNA-nucleic acid linker-protein complex, in which the C-terminal of the protein is bound to a protein linking segment of the nucleic acid linker, by synthesizing the protein from the mRNA using a cell-free protein translation system.

The following provides an explanation of each step.

In step (a), the mRNA and the nucleic acid linker are annealed. First, an explanation is provided of preparation of the mRNA used in step (a).

The mRNA is obtained by preparing DNA encoding a protein to be screened and transcribing with RNA polymerase. An example of RNA polymerase is T7 RNA polymerase.

A DNA or DNA library encoding an arbitrary protein desired to be investigated with respect to bonding with a target molecule can be used for the aforementioned DNA. Examples thereof that can be used include a cDNA library obtained from a sample tissue, a DNA library obtained by random sequence synthesis, and a DNA library obtained by partial sequence mutation.

The 3′-side of mRNA following transcription is designed so as to hybridize with the single-stranded polynucleotide segment of the nucleic acid linker of the present embodiment by inserting a common tag sequence into the 3′-terminal of the DNA prior to transcription.

Next, the 3′-terminal region of the mRNA and the single-stranded polynucleotide segment of the nucleic acid linker of the present embodiment are annealed. For example, the mRNA can be reliably hybridized to the nucleic acid linker by denaturing the mRNA by heating to 90° C. followed by cooling to 25° C. over the course of 15 minutes.

Next, in step (b), the 3′-terminal of the mRNA and one region of the 5′-terminal region of the nucleic acid linker are ligated. During ligation, it is necessary to phosphorylate the 5′-terminal of one region of the 5′-terminal region using an enzyme such as T4 polynucleotide kinase. An RNA ligase is preferably used for the enzyme used for ligation, and an example thereof is T4 RNA ligase.

Next, in step (c), the mRNA-nucleic acid linker-protein complex is prepared, in which the C-terminal of the protein is bound to the protein linking segment of the nucleic acid linker, by synthesizing the protein from the mRNA using a cell-free protein translation system.

A cell-free protein translation system refers to a protein translation system composed of components having the ability to synthesize protein that have been extracted from suitable cells, and elements required for translation are contained in this system, examples of which include ribosomes, translation initiation factors, translation elongation factors, dissociating factors and aminoacyl-tRNA synthetase. Examples of such protein translation systems include Escherichia coli extract, rabbit reticulocyte extract and wheat germ extract.

Moreover, another example of a cell-free protein translation system is a reconstituted cell-free protein synthesis system composed only of factors in which elements required for translation have been independently purified. Reconstituted cell-free protein synthesis systems are able to enhance translation efficiency since they are able to more easily prevent contamination by nucleases or proteases than in the case of using conventional cell extracts.

The mRNA-nucleic acid linker-protein complex is produced by using such a system.

<<mRNA/cDNA-Nucleic Acid Linker-Protein Complex>>

A method for producing an mRNA/cDNA-nucleic acid linker-protein complex has a step (d) in addition to the steps comprising the previously described method for producing an mRNA-nucleic acid linker-protein complex.

Step (d) is a step for synthesizing cDNA from the previously described the mRNA-nucleic acid linker-protein complex by reverse transcription. A known reverse transcriptase is used for the reverse transcriptase used in reverse transcription, and an example thereof is reverse transcriptase derived from Moloney murine leukemia virus.

The reverse transcribed cDNA forms a hybrid with the mRNA of the mRNA-nucleic acid linker-protein complex. In addition to the mRNA in the mRNA-nucleic acid linker-protein complex being more easily degradable than cDNA, since it also has a high possibility of non-specifically interacting as aptamers, in the case of analyzing protein interaction, it is preferable to prepare this type of mRNA/cDNA-nucleic acid linker-protein complex.

In addition, it is also essential to prepare this complex in order to analyze cDNA that encodes a protein which has been found to be useful as a result of screening.

<<Protein Array>>

A protein array is produced by immobilizing the previously described protein complex on a microarray substrate. Examples of substrates used include a glass substrate, silicon substrate, plastic substrate and metal substrate. Since a solid phase binding site is provided in the protein complex, the protein complex is immobilized on the microarray substrate by utilizing binding between that solid phase binding site and a solid phase binding site recognition site bound to the substrate.

In addition to the use of avidin-biotin bonding, examples of methods that can be used to immobilize the nucleic acid linker when using a combination of a solid phase binding site and a solid phase binding site recognition site include a method consisting of modifying the nucleic acid linker with a functional group such as an amino group, formyl group or SH group and treating the surface of the solid phase with a silane coupling agent having an amino group, formyl group or epoxy group and the like, and a method that utilizes gold-thiol bonding, while a method that utilizes avidin-biotin bonding is particularly preferable.

Although the following provides an explanation of the present invention using examples thereof, the present invention is not limited to the following examples.

### EXAMPLES

### [Synthesis of Nucleic Acid Linker—1]

1-1 Materials

Synthesis of the two types of DNA oligomers indicated below was commissioned to JBioS, and the DNA oligomers were synthesized in accordance with the phosphoramidite method using an automated nucleic acid synthesizer.

(1) dl-Branch-Thiol Segment

[Sequence: 5′-(B)-(spc18)-AAAAA-(dI)-AAAAA-(C-CCC-
5′)-X1-(T-NH₂)-CCT-3′]

X1 represents the sequence indicated below.

| | |
|---|---|
| CCCCGCCGCCCCCCG | (SEQ ID NO: 1, 15 mer) |

(2) Puromycin Segment

[Sequence: 5′-(HO-C₆H₁₂-SS-C₆H₁₂)-TC(F)-(spc18)-
(spc18)-(spc18)-CC-(Puromycin)-3′]

Here, (B) represents that synthesized using [1-N-(4,4′-dimethoxytrityl)-biotinyl-6-aminohexyl]-2-cyanoethyl-(N,N-diisopropyl)-phosphoramidite (trade name: 5′-Biotin Phosphoramidite, Glen Research).

(F) represents that synthesized using 5′-dimethyloxytrityloxy-5-[N-[(3′,6′-dipivaloylfluoresceinyl)-aminohexyl]-3-acryimido]-2′-deoxyuridine-3′-succinoyl-long chain alkylamino (trade name: Fluorescein-dT, Glen Research).

(spc18) represents that synthesized using 18-O-dimethoxytritylhexaethylene glycol, 1-[(2-cyanoethyl)-(N,N-diisopropyl)]-phosphoramidite (trade name: Spacer Phosphoramidite 18, Glen Research).

(dI) indicates deoxyinosine, and represents that synthesized using 5′-dimethoxytrityl-2′-deoxyinosine, 3′-[(2-cyanoethyl)-(N,N-diisopropyl)]-phosphoramidite (trade name: dI-CE phosphoramidite, Glen Research).

(C—CCC-5′) represents that obtained by condensing deoxycytosine by three bases in the 3′→5′ direction in the base side branch using 5′-dimethoxytrityl-N4-(O-levulinyl-6-oxyhexyl)-5-methyl-2′-deoxycytidine, 3′-[(2-cyanoethyl)-(N,N-diisopropyl)]-phosphoramidite (trade name: 5-Me-dC Brancher Phosphoramidite, Glen Research).

(T-NH₂) represents that synthesized using 5′-dimethoxytrityl-5-[N-(trifluoroacetylaminohexyl)-3-acrylimido]-2′-deoxyuridine, 3′-[(2-cyanoethyl)-(N,N-diisopropyl)]-phosphoramidite (trade name: Amino-Modifier C6 dT, Glen Research).

(HO—C₆H₁₂—SS—C₆H₁₂) represents that synthesized using (1-O-dimethyoxytrityl-hexyl-disulfide, 1′-[(2-cyanoethyl)-(N,N-diisopropyl)]-phosphoramidite (trade name: Thiol-Modifier C6 S—S, Glen Research).

(Puromycin) represents that synthesized using 5′-dimethoxytrityl-N-trifluoroacetyl-puromycin, 2′-succinoyl-long chain alkylamino-CPG (trade name: Puromycin-CPG, Glen Research).

1-2 Synthesis and Purification Methods

(Synthesis of mRNA)

The B-domain of Protein A (to be referred to as BDA, SEQ ID NO: 2, 367 bp), obtained by adding a T7 promoter sequence and translation promoting sequence upstream from the 5′-side and adding a spacer region and sequence having a complementary strand region with the dI-Branch-Biotin segment downstream from the 3′-side, was amplified by PCR.

5 pmol/μl to 30 pmol/μl mRNA (337 b) was synthesized from the DNA obtained by PCR using the T7 RiboMAX Express Large Scale RNA Production System (Promega) in accordance with the protocol provided.

(Ligation and Reverse Transcription of dI-Branch-Biotin Segment and mRNA)

20 pmol of the aforementioned mRNA and 40 pmol of the aforementioned dI-Branch-Biotin segment were mixed in 19 μl of T4 RNA Ligase buffer (Takara Bio) and heated to 90° C. followed by cooling to 25° C. over the course of 15 minutes. 0.5 μl of T4 polynucleotide kinase (10 U/μl, Toyobo) and 0.5 μl of T4 RNA ligase (40 U/μl, Takara Bio) were added to this solution and mixed therein followed by reacting for 15 minutes at 25° C.

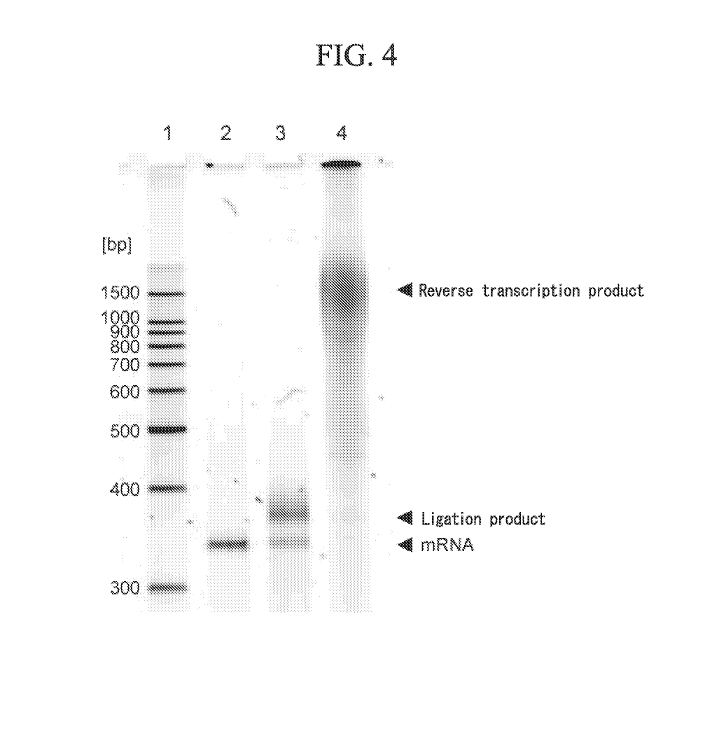

The reaction product was separated by 8 M urea/5% polyacrylamide gel electrophoresis (200 V, 60° C., 60 minutes) and stained with SybrGold (Invitrogen). The results are shown in FIG. 4.

Lane 1 is a 100 bp DNA ladder (Promega), lane 2 is mRNA (BDA), lane 3 is the ligation product of the dI-Branch-Biotin segment and mRNA (BDA), and lane 4 is the reverse transcription product of the aforementioned ligation product.

The dI-Branch-Biotin segment linked with the mRNA, and the band was able to be observed to shift towards the high molecular weight side, thereby confirming that the synthesized nucleic acid linker has the ability to link with mRNA.

Moreover, the aforementioned ligation product was purified using the RNeasy MiniElute Cleanup Kit (Qiagen). 2 pmol of this ligation product, 4 μl of 2.5 mM dNTP Mix (Takara Bio), 2 μl of 5×RT buffer (Toyobo), 0.5 μl of ReverTraAce (100 U/μl, Toyobo) and RNase-free water were mixed to obtain 10 μl of a mixture. This mixture was then allowed to react for 30 minutes at 42° C. to obtain a reverse transcription product. This reverse transcription product was separated by 8 M urea/5% polyacrylamide gel electrophoresis (200 V, 60° C., 60 minutes) and stained with SybrGold (Invitrogen). The results are shown in FIG. 4.

In lane 4, the band shifted farther towards the high molecular weight side than the ligation product shown in lane 3, thereby confirming that reverse transcription had been carried out.

(Confirmation of Solid Phase Binding Ability and Cleavage Susceptibility of dI-Branch-Biotin Linker)

2 μl of 7.65 μM dI-Branch-Biotin segment and 2 μl of 2 μM streptavidin (Sigma) dissolved in 0.1M PBS were mixed and allowed to stand undisturbed for 10 minutes at room temperature.

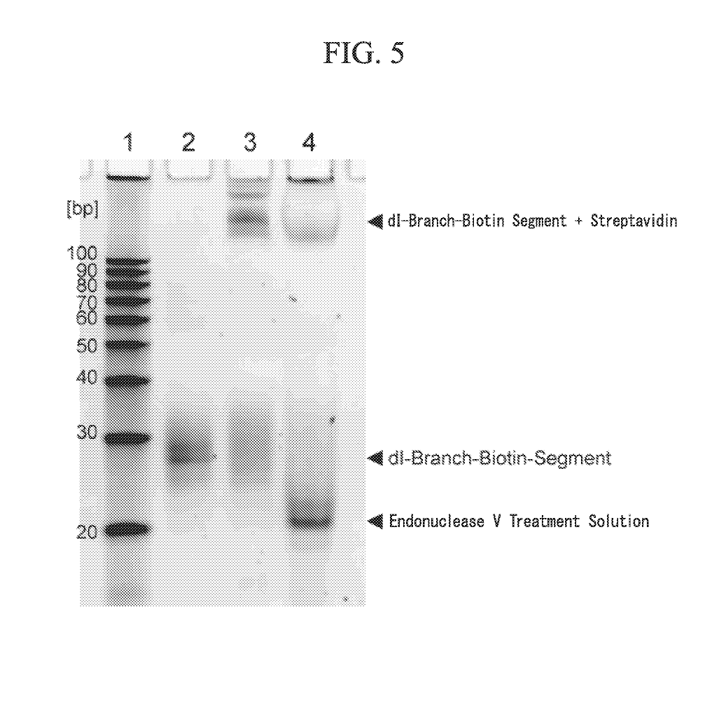

10×NE Buffer 4 (New England BioLabs), Endonuclease V (1 U/μl, New England BioLabs) and RNase-free water were mixed with 2 μl of this mixture to obtain 5 μl of a mixture. This mixture was allowed to react for 10 minutes at 37° C. The reaction product was isolated by 12% polyacrylamide gel electrophoresis (200 V, 30° C., 30 minutes) and stained with SybrGold (Invitrogen). The results are shown in FIG. 5.

Lane 1 is a 100 bp DNA ladder (Promega), lane 2 is the dI-Branch-Biotin segment, lane 3 is the mixture of the dI-Branch-Biotin segment and streptavidin, and lane 4 is the Endonuclease V treatment solution.

It can be understood from lane 3 that the dI-Branch-Biotin segment bound to streptavidin shifted towards the high molecular weight side. Accordingly, the dI-Branch-Biotin segment was confirmed to have the ability to bind to a solid phase through biotin.

It can be understood from lane 4 that the DNA strand was cleaved by Endonuclease V in the vicinity of deoxyinosine, and that the cleaved fragment of the dI-Branch-Biotin segment desorbed from the streptavidin and shifted towards the low molecular weight side. Accordingly, the dI-Branch-Biotin segment was confirmed to be cleaved by Endonuclease V and have the ability to desorb from a solid phase.

(Reduction of Puromycin Segment)

0.8 μl of 3 mM Puromycin segment and 11.3 μl of 1 M phosphate buffer (pH 9.0) were mixed followed by the addition of 1.25 μl of 1 M DTT and reacting for 1 hour at room temperature to reduce the disulfide group on the 5′-side of the Puromycin segment to a thiol group. Subsequently, excess DTT was removed using an NAP-5 column (GE Healthcare Japan) equilibrated with 20 mM phosphate buffer (pH 7.2).

(EMCS Modification of dI-Branch-Thiol Segment)

1.6 μl of 0.77 mM dI-Branch-Biotin segment were mixed with 25 μl of 0.2 M phosphate buffer (pH 7.2) followed by the addition of 5 μl of 0.1 M divalent crosslinking agent EMCS (6-maleimidohexanoic acid N-hydroxysuccinimide ester, Dojindo Laboratories), stirring well and reacting for 30 minutes at 37° C. Subsequently, the reaction product was precipitated by ethanol precipitation followed by removal of unreacted EMCS. The precipitate was washed with 200 μl of 70% ethanol.

(Crosslinking of Puromycin Segment and dI-Branch-Biotin Segment)

The precipitate of the aforementioned EMCS-crosslinked dI-Branch-Biotin segment was dissolved in a solution of the aforementioned reduced Puromycin segment and allowed to stand overnight at 4° C. Next, the crosslinking reaction was stopped by adding and mixing in 10 μl of 1 M DTT followed by stirring for 30 minutes at room temperature.

Subsequently, the reaction product was precipitated by ethanol precipitation, and after removing the unreacted Puromycin segment and excess DTT and washing the precipitate with 200 μl of 70% ethanol, the precipitate was dissolved in 15 μl of sterile water and adjusted to a concentration of 45 μM. The resulting crosslinked product was separated by 8 M urea/12% polyacrylamide gel electrophoresis (200 V, 60° C., 30 minutes) followed by staining with SybrGold (Invitrogen).

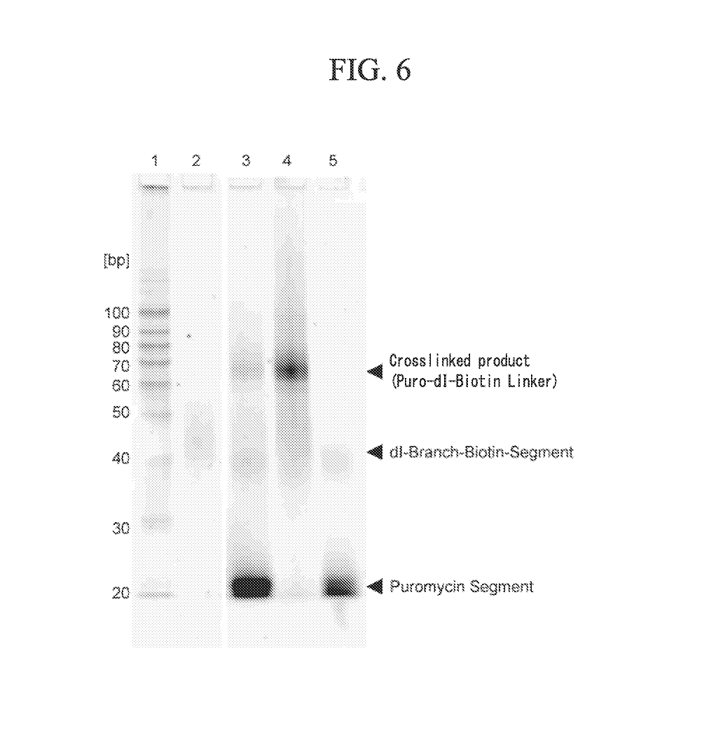

The results are shown in FIG. 6. Lane 1 is a 10 bp DNA step ladder (Promega), lane 2 is the dI-Branch-Biotin segment, Lane 3 is the crosslinked product of the Puromycin segment and the dI-Branch-Biotin segment, lane 4 is the crosslinked product purified by ethanol precipitation, and lane 5 is the supernatant obtained following ethanol precipitation of the crosslinked product. It was confirmed from lane 4 that the target crosslinked product (Puro-dI-Biotin linker) was obtained.

(Ligation of Puro-dI-Biotin Linker and mRNA)

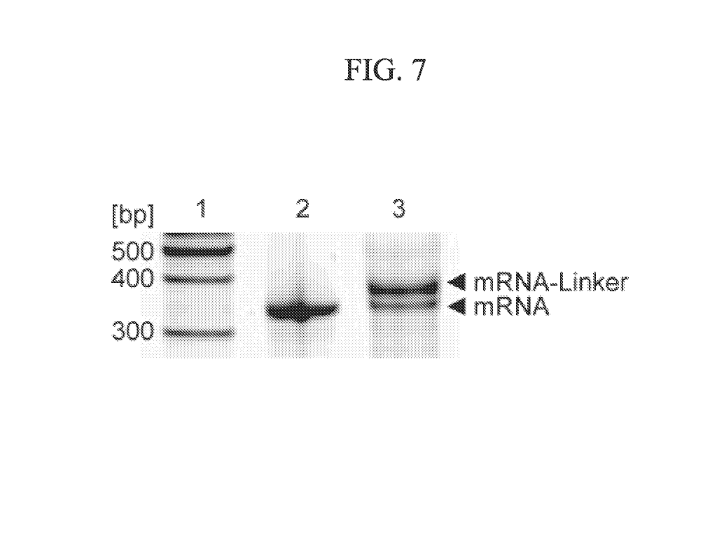

20 pmol of BDA mRNA synthesized according to the method previously described in the section entitled “Synthesis of mRNA” and 40 pmol of the aforementioned Puro-dI-Biotin linker were mixed in 18 μl of T4 RNA Ligase buffer (Takara Bio) and heated to 90° C. followed by cooling to 25° C. over the course of 15 minutes. 1 μl of T4 polynucleotide kinase (10 U/μl, Toyobo) and 1 μl of T4 RNA ligase (40 U/μl, Takara Bio) were added to this solution and mixed therein followed by reacting for 15 minutes at 25° C. The reaction product was separated by 8 M urea/8% polyacrylamide gel electrophoresis (200 V, 60° C., 40 minutes) and stained with SybrGold (Invitrogen). The results are shown in FIG. 7.

Lane 1 is a 100 bp DNA ladder (Promega), lane 2 is mRNA (BDA), and lane 3 is the ligation product of the Puro-dI-Biotin linker and mRNA (BDA).

The Puro-dI-Biotin linker and mRNA were observed to ligate and the band was observed to shift towards the high molecular weight side. Namely, the nucleic acid linker of the present embodiment was confirmed to have the ability to ligate with mRNA.

(Protein Display Using Puro-dI-Biotin Linker)

A translation reaction was carried out using the nucleic acid linker (Puro-dI-Biotin linker) and mRNA ligation product synthesized in the manner described above. RNase-free water was added and mixed with 1 pmol of mRNA-nucleic acid linker ligation product (mRNA-Linker ligation product), 0.72 μl of 20× Translation Mix (Ambion), and 10.2 μl of rabbit reticulocyte cell lysate in the form of Rabbit Retic Lysate (Ambion) to obtain 15 μl of a mixture.

After allowing this mixture to react for 20 minutes at 30° C., 6 μl of 3 M calcium chloride solution and 1.8 μl of 1 M magnesium chloride solution were added and mixed therein. This mixture was then allowed to react for 30 minutes at 37° C. to synthesize a polypeptide chain of BDA gene and form an mRNA-nucleic acid linker-protein complex. The reaction product was separated by SDS containing 8 M urea/6% polyacrylamide gel electrophoresis, and the fluorescence signal of the fluorescein used to modify the nucleic acid linker was detected.

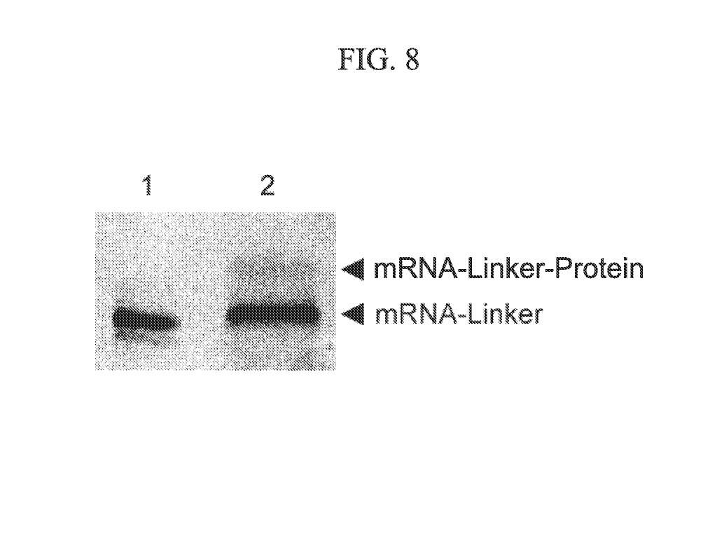

The results are shown in FIG. 8.

Lane 1 is the mRNA-linker ligation product, and lane 2 is the translation product.

According to the results of electrophoresis, since a band of the mRNA-linker ligation product was detected in lane 2 that shifted towards the high molecular weight side, the nucleic acid linker of the present embodiment was confirmed to have the ability to display protein.

[Synthesis of Nucleic Acid Linker—2]

2-1 Materials

Synthesis of the three types of DNA oligomers indicated below was commissioned to JBioS, and the DNA oligomers were synthesized in accordance with the phosphoramidite method using an automated nucleic acid synthesizer.

(1) PC-Branch-Thiol Segment

[Sequence: 5′-(HO-C₆H₁₂-SS-C₆H₁₂)-
TTTTTTTTTTTTTTTTTTTTTTTTTTTTTT-(PC)-TTT(C-
CCC-5′)-X1-(T-NH₂)-CCT-3′]

X1 is as previously defined.

(2) PC-Branch-Biotin Segment

[Sequence: 5′-(B)-TTTTTTTTTTTTTTTTTTTT-(PC)-
TTT(C-CCC-5′)-X1-(T-NH₂)-CCT-3′]

X1 is as previously defined.

(3) Puromycin Segment

[Sequence: 5′-(HO-C₆H₁₂-SS-C₆H₁₂)-TCT-(spc18)-
(spc18)-(spc18)-CC-(Puromycin)-3′]

Here, (HO—C₆H₁₂—SS—C₆H₁₂), (C—CCC-5′), (T-NH₂), (B), (spc18) and (Puromycin) are as previously defined.

(PC) represents that synthesized using [4-(4,4′-dimethoxytrityloxy)butyramidomethyl]-1-(2-nitrophenyl)-ethyl]-2-cyanoethyl-(N,N-diisopropyl)-phosphoramidite (trade name: PC Spacer Phosphoramidite, Glen Research).

2-2 Synthesis and Purification Methods

(Reduction of Puromycin Segment)

18 μl of 2.5 mM Puromycin segment and 90 μl of 1 M phosphate buffer (pH 9.0) were mixed followed by the addition of 10 μl of 1 M DTT and reacting for 1 hour at room temperature to reduce the disulfide group on the 5′-side of the Puromycin segment to a thiol group. Subsequently, excess DTT was removed using an NAP-5 column (GE Healthcare Japan) equilibrated with 20 mM phosphate buffer (pH 7.2).

(EMCS Modification of PC-Branch-Thiol Segment)

10 μl of 1 mM PC-Branch-Thiol segment were mixed with 100 μl of 0.2 M phosphate buffer (pH 7.2) followed by the addition of 20 μl of 0.1 M divalent crosslinking agent EMCS (6-maleimidohexanoic acid N-hydroxysuccinimide ester, Dojindo Laboratories), stirring well and reacting for 30 minutes at 37° C. Subsequently, the reaction product was precipitated by ethanol precipitation followed by removal of unreacted EMCS. The precipitate was washed with 200 μl of 70% ethanol.

(Crosslinking of Puromycin Segment and PC-Branch-Thiol Segment or PC-Branch-Biotin Segment)

The precipitate of the aforementioned EMCS-crosslinked PC-Branch-Thiol segment or the precipitate of the aforementioned EMCS-crosslinked PC-Branch-Biotin segment was dissolved in a solution of the aforementioned reduced Puromycin segment (approx. 20 nmol) and allowed to stand overnight at 4° C.

Subsequently, the reaction product was precipitated by ethanol precipitation. After washing the precipitate with 200 μl of 70% ethanol, the precipitate was dissolved in 30 μl of sterile water. The resulting crosslinked product was separated by 8 M urea/12% polyacrylamide gel electrophoresis followed by staining with SybrGold (Invitrogen).

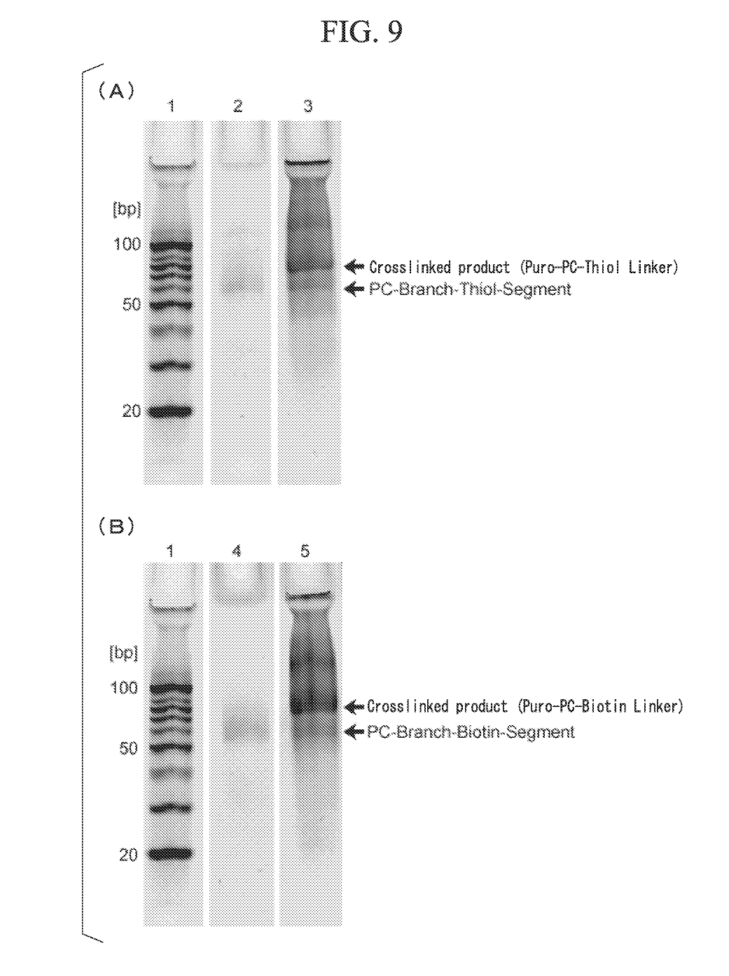

The results are shown in FIG. 9. Lane 1 is a 10 bp DNA step ladder (Promega), lane 2 is the PC-Branch-Thiol segment, Lane 3 is the crosslinked product of the PC-Branch-Thiol segment and the Puromycin segment, lane 4 is the PC-Branch-Biotin segment, and lane 5 is the crosslinked product of the PC-Branch-Biotin segment and Puromycin segment. The target crosslinked products (Puro-PC-Thiol linker and Puro-PC-Biotin linker) were confirmed to be obtained from lanes 3 and 5.

(HPLC Purification of Puro-PC-Thiol Linker and Puro-PC-Biotin Linker)

The Puro-PC-Thiol linker and Puro-PC-Biotin linker synthesized in the manner described above were purified by HPLC.

(Synthesis of mRNA-Nucleic Acid Linker Complex)

5 pmol of BDA mRNA synthesized according to the previously described method and 10 pmol of the Puro-PC-Thiol linker or 10 pmol of the Puro-PC-Biotin linker were mixed in T4 RNA Ligase buffer (Takara Bio) and heated to 90° C. followed by cooling to 25° C. over the course of 15 minutes. 0.5 μl of T4 polynucleotide kinase (10 U/μl, Toyobo) and 0.5 μl of T4 RNA ligase (40 U/μl, Takara Bio) were added to this solution and mixed therein followed by reacting for 15 minutes at 25° C.

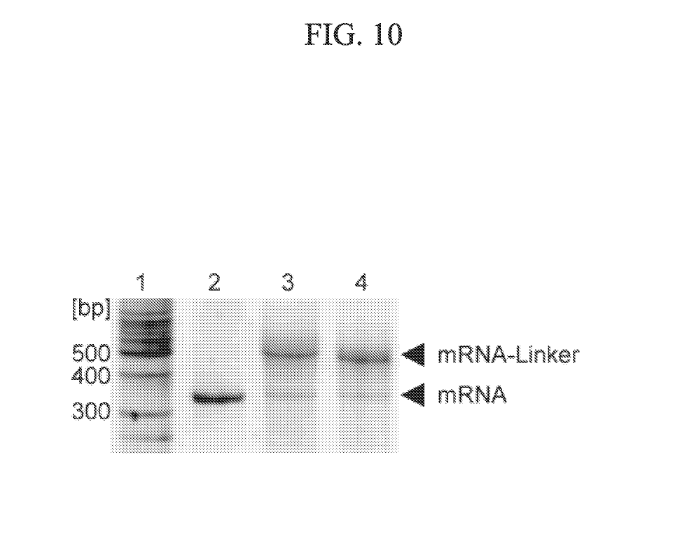

The reaction product was separated by 8 M urea/8% polyacrylamide gel electrophoresis and stained with SybrGold (Invitrogen). The results are shown in FIG. 10.

Lane 1 is a 100 bp DNA ladder (Promega), lane 2 is mRNA (BDA), lane 3 is the ligation product of the Puro-PC-Thiol linker and mRNA (BDA), and lane 4 is the ligation product of the Puro-PC-Biotin linker and mRNA (BDA).

Both the Puro-PC-Thiol linker and Puro-PC-Biotin linker linked with the mRNA and the bands were able to be observed to shift towards the high molecular weight side, thereby confirming that the synthesized nucleic acid linkers have the ability to link with mRNA.

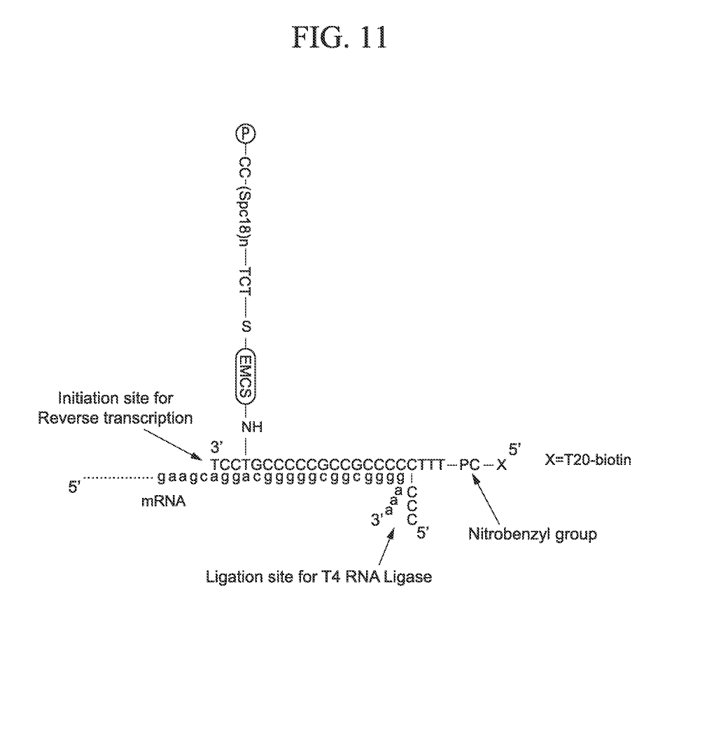

FIG. 11 shows a schematic diagram of the hybridization product of mRNA (BDA) and a nucleic acid linker. In FIG. 11, P indicates puromycin and PC indicates a photocleavage site (nitrobenzyl group). Upper case letters indicate the DNA segment while lower case letters indicate the mRNA segment. X indicates 5′-(B)-TTTTTTTTTTTTTTTTTTTT-3′.

(Translation by Cell-Free Translation System)

Translation reactions were carried out using the nucleic acid linker and mRNA ligation products synthesized in the manner described above. RNase-free water was added and mixed with 1 pmol of mRNA-nucleic acid linker ligation product (mRNA-Linker ligation product), 0.72 μl of 20× Translation Mix (Ambion), 10.2 μl of rabbit reticulocyte cell lysate in the form of Rabbit Retic Lysate (Ambion) and 0.3 μl of Fluorotect (Promega) to obtain 15 μl of a mixture.

After allowing this mixture to react for 20 minutes at 30° C., 6 μl of 3 M calcium chloride solution and 1.8 μl of 1M magnesium chloride solution were added and mixed therein. This mixture was further allowed to react for 30 minutes at 37° C. to synthesize a polypeptide chain of BDA gene and form an mRNA-nucleic acid linker-protein complex. The reaction product was separated by SDS containing 8 M urea/6% polyacrylamide gel electrophoresis, and the fluorescence signal of Fluorotect incorporated in the protein was detected.

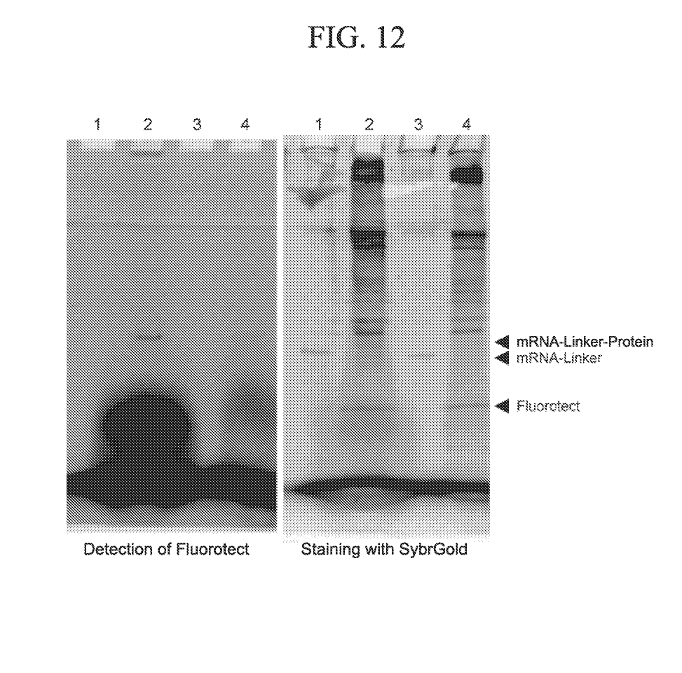

Moreover, mRNA was detected by staining the reaction product with SybrGold (Invitrogen). The results are shown in FIG. 12.

Lane 1 is the ligation product of the Puro-PC-Thiol linker and mRNA (BDA), lane 2 is the translation product of the ligation product of the Puro-PC-Thiol linker and mRNA (BDA), lane 3 is the ligation product of the Puro-PC-Biotin linker and mRNA (BDA), and lane 4 is the translation product of the ligation product of the Puro-PC-Biotin linker and mRNA (BDA).

According to the results of electrophoresis, bands of the mRNA-protein complex were able to be confirmed that demonstrated a fluorescence signal farther to the high molecular weight side than mRNA, thereby confirming that the synthesized nucleic acid linkers of the present embodiment have the ability to display protein.

On the basis of the above results, it is clear that the nucleic acid linker of the present embodiment is optimally suited for immobilization on a solid phase while also enabling highly functional molecular manipulation.

### INDUSTRIAL APPLICABILITY

An object of embodiments of the present invention is to provide a nucleic acid linker that is optimally suited for immobilization on a solid phase while also enabling highly functional molecular manipulation.

Since the nucleic acid linker of embodiments of the present invention is optimally suited for immobilization on a solid phase while also enabling highly functional molecular manipulation, it is preferably used for comprehensive analysis.

What is claimed is:

1. A nucleic acid linker for producing a complex of mRNA and a protein encoded by that mRNA, comprising:
one 3′-terminal region, and
two branched 5′-terminal regions; wherein,
the 3′-terminal region comprises a single-stranded polynucleotide segment able to hybridize with the sequence on the 3′-terminal side of the mRNA, and
an arm segment that branches off from the single-stranded polynucleotide segment and has a linking segment with the protein on the terminal thereof, and
one of the two 5′-terminal regions has a binding site with the 3′-terminal of the mRNA.

2. The nucleic acid linker according to claim 1, wherein the other of the two 5′-terminal regions has a solid phase binding site on the 5′-terminal thereof.

3. The nucleic acid linker according to claim 1, wherein the other of the two 5′-terminal regions contains a cleavage site.

4. The nucleic acid linker according to claim 1, wherein one of the 3′-terminal region and the 5′-terminal region contains a cleavage site.

5. The nucleic acid linker according to claim 1, wherein the linking segment with the protein has puromycin, a 3′-N-aminoacyl puromycin aminonucleoside or 3′-N-aminoacyl adenosine aminonucleoside bound to the end of the arm segment.

6. The nucleic acid linker according to claim 1, wherein one of the two 5′-terminal regions and the 3′-terminal region form a loop region.

7. The nucleic acid linker according to claim 6, wherein one of the two 5′-terminal regions and the 3′-terminal region respectively contain a cleavage site.

8. A nucleic acid linker for producing a complex of mRNA and a protein encoded by that mRNA, provided with:
a 3′-terminal region containing a single-stranded polynucleotide segment able to hybridize with the sequence on the 3′-terminal side of the mRNA,
two branched 5′-terminal regions, and
an arm segment having a linking segment with the protein on the terminal thereof; wherein,
at least one of the two 5′-terminal regions has a spacer region containing a solid phase binding site on the 5′-terminal thereof.

9. The nucleic acid linker according to claim 8, wherein the protein composes any one of an enzyme, antibody, antigen, aptamer and peptide.

10. The nucleic acid linker according to claim 1, wherein the arm segment has a labeling substance.

11. An mRNA-nucleic acid linker-protein complex obtained by linking the mRNA and a protein encoded by that mRNA through the nucleic acid linker according to claim 1.

12. An mRNA/cDNA-nucleic acid linker-protein complex obtained by linking an mRNA/cDNA complex, composed of the mRNA and cDNA complementary to the mRNA, and a protein encoded by that mRNA through the nucleic acid linker according to claim 1.

13. A method for producing the mRNA-nucleic acid linker-protein complex according to claim 11, having:
(a) a step for annealing the mRNA and the nucleic acid linker,
(b) a step for ligating the 3′-terminal of the mRNA and the 5′-terminal of the nucleic acid linker, and
(c) a step for preparing the mRNA-nucleic acid linker-protein complex, in which the C-terminal of the protein is bound to a linking segment with the protein of the nucleic acid linker, by synthesizing the protein from the mRNA using a cell-free protein translation system.

14. A method for producing the mRNA/cDNA-nucleic acid linker-protein complex according to claim 12, having:
(a) a step for annealing the mRNA and the nucleic acid linker,
(b) a step for ligating the 3′-terminal of the mRNA and the 5′-terminal of the nucleic acid linker,
(c) a step for preparing an mRNA-nucleic acid linker-protein complex, in which the C-terminal of the protein is bound to a linking segment with the protein of the nucleic acid linker, by synthesizing the protein from the mRNA using a cell-free protein translation system, and
(d) a step for synthesizing cDNA from the mRNA-nucleic acid linker-protein complex by reverse transcription.

15. A protein array obtained by immobilizing the protein complex according to claim 11 on a substrate.
